(19) United States
(12) Patent Application Publication
(10) Pub. No.: US 2014/0094843 A1
(43) Pub. Date: Apr. 3, 2014

(54) EMBOLISM PROTECTION DEVICE

(71) Applicant: Claret Medical, Inc., Santa Rosa, CA (US)

(72) Inventor: Richard R. Heuser, Phoenix, AZ (US)

(21) Appl. No.: 14/036,532

(22) Filed: Sep. 25, 2013

Related U.S. Application Data
(63) Continuation of application No. 13/449,165, filed on Apr. 17, 2012, now abandoned.
Application No. 13/449,165 is a continuation-in-part of application No. 12/905,056, filed on Oct. 14, 2010.

(51) Int. Cl. A61B 17/12

(52) U.S. Cl. CPC A61B 17/12109
USPC 606/200

(57) ABSTRACT

Disclosed herein are systems and methods for protecting a subject from embolisms during TAVI and other percutaneous valve procedures. Some embodiments include two flexible catheters designed to pass over a guidewire into two separate arteries, such as carotid or innominate arteries, each flexible catheter including a vessel blocking mechanism and a bypass feature that prevents ischemia while the vessel blocking mechanism is in use. Other embodiments include a single, bifurcated flexible catheter having two arms, each of which is configured to protect a different artery. In some embodiments, each arm includes a vessel blocking mechanism and a bypass feature that prevents ischemia while the vessel blocking mechanism is in use. In some embodiments, the systems also may include one or more concentric guidewires.

### INCORPORATION BY REFERENCE TO ANY PRIORITY APPLICATIONS

The present application is a continuation of U.S. patent application Ser. No. 13/449,165, filed Apr. 17, 2012, entitled EMBOLISM PROTECTION DEVICE, which is a continuation-in-part of U.S. patent application Ser. No. 12/905,056, filed Oct. 14, 2010, entitled CONCENTRIC WIRE EMBOLISM PROTECTION DEVICE, the disclosure of which is hereby incorporated by reference in its entirety.

The present application is also related to U.S. patent application Ser. No. 12/140,183, filed Jan. 8, 2009, entitled “CATHETER GUIDEWIRE SYSTEM USING CONCENTRIC WIRES;” and U.S. Pat. No. 7,402,141, issued Jul. 22, 2008, entitled “CATHETER GUIDEWIRE SYSTEM USING CONCENTRIC WIRES,” the disclosures of which are hereby incorporated by reference in their entirety.

### BACKGROUND OF THE INVENTION

1. Field of the Invention

Embodiments relate to an embolism-preventing device that prevents the free flow of embolism-creating particles that are created during cardiac procedures such as percutaneous valve interventions, particularly transcatheter aortic valve implantation (TAVI) procedures.

2. Description of the Related Art

An embolus can be any particle comprising a foreign or native material that enters the vascular system with potential to cause occlusion of blood flow. Emboli can be formed from aggregated fibrin, red blood cells, collagen, cholesterol, plaque, fat, calcified plaque, bubbles, arterial tissue, and/or other miscellaneous fragments. Each dislodged fragment, or embolus, is carried along by the blood flow until it becomes lodged or trapped in a smaller vessel and occludes blood flow, creating an embolism. Since emboli reduce or cut off blood flow, damage to the body may result, such as tissue damage, heart attack, stroke, or even death.

Percutaneous valve interventions include valvuloplasty, annuloplasty, and valve replacement surgeries performed on the mitral, tricuspid, aortic, and pulmonary valves. These interventions carry a high risk of embolism formation. For instance, aortic valve applications, such as transcatheter aortic valve implantation (TAVI) procedures, may carry a 60-80% chance of embolism formation and subsequent cerebral ischemic events.

### BRIEF DESCRIPTION OF THE DRAWINGS

Embodiments will be readily understood by the following detailed description in conjunction with the accompanying drawings. Embodiments are illustrated by way of example and not by way of limitation in the figures of the accompanying drawings.

FIG. 1 illustrates a cross-sectional side view of an embodiment of the guidewire system used to deploy the embolism protection devices disclosed herein, showing three concentric wires, including the proximal and distal ends, central lumens, and proximal handles, in accordance with various embodiments;

FIG. 2 illustrates an example of an embolism protection device that has been positioned over a guidewire and inside a sheath in a desired artery;

FIG. 3 illustrates the device shown in FIG. 2 after the sheath has been retracted to expose a flexible catheter having a vessel blocking mechanism coupled thereto;

FIG. 4 illustrates a bifurcated flexible catheter system having two arms after it has been deployed in two separate arteries;

FIG. 5 illustrates the bifurcated flexible catheter system shown in FIG. 4 after the vessel blocking mechanisms have been activated;

FIG. 6 illustrates an embolism protection system having two flexible catheters deployed in different arteries; and

FIG. 7 illustrates a port for use with the disclosed embolism protection systems and devices, all in accordance with various embodiments.

### DETAILED DESCRIPTION OF THE PREFERRED EMBODIMENT

In the following detailed description, reference is made to the accompanying drawings which form a part hereof, and in which are shown by way of illustration embodiments that may be practiced. It is to be understood that other embodiments may be utilized and structural or logical changes may be made without departing from the scope. Therefore, the following detailed description is not to be taken in a limiting sense, and the scope of embodiments is defined by the appended claims and their equivalents.

Various operations may be described as multiple discrete operations in turn, in a manner that may be helpful in understanding embodiments; however, the order of description should not be construed to imply that these operations are order dependent.

The description may use perspective-based descriptions such as up/down, back/front, and top/bottom. Such descriptions are merely used to facilitate the discussion and are not intended to restrict the application of disclosed embodiments.

The terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element.

The description may use the terms “embodiment” or “embodiments,” which may each refer to one or more of the same or different embodiments. Furthermore, the terms “comprising,” “including,” “having,” and the like, as used with respect to embodiments, are synonymous.

Embodiments herein provide embolism protection devices that may be deployed in a body vessel for the collection of loosened or floating debris, such as embolic material dislodged during or thrombi formed as a result of percutaneous cardiac procedure, such as a valve intervention. In various embodiments, the device may be deployed in the left and/or right carotid or innominate arteries (or both the left and right carotid and/or innominate arteries) to protect the subject from brain embolism associated with, for instance, percutaneous treatment or replacement of the aortic or mitral valve, or any coronary stent or bypass procedure, and may be particularly useful for TAVI procedures. In other embodiments, the device may be used to protect the vasculature of a patient from dislodged materials (e.g., potential emboli and thrombi) from valvular or coronary artery disease, or during angioplasty, atherectomy, thrombectomy, embolectomy, intravascular diagnostic procedures, and/or stent placement procedures. Embodiments of the device also may be used to protect the patient from emboli and thrombi resulting from open interventional procedures including transapical approaches to treat valvular disease or any minimally invasive heart intervention.

In various embodiments, the device may include one or more flexible catheters that may be inserted along one or more guidewires and passed into one or more arteries, such as a left or right innominate or carotid artery (or both). In various embodiments, once each catheter is positioned in a desired location, a vessel blocking mechanism, such as a compliant expandable membrane configured to be atraumatic to the vessel, for instance an elastomeric membrane or very compliant balloon, coupled to an outside surface of each flexible catheter, may be inflated in order to prevent the free flow of blood (which may carry material that could form emboli). In various embodiments, a plurality of perforations may be provided both distal and proximal to the vessel blocking mechanism, and these perforations may be sized and spaced to allow fluid (e.g., blood) to bypass the vessel blocking mechanism without allowing particulates to pass that may form embolisms. Some embodiments may use two catheters in two separate arteries, whereas other embodiments may use a single bifurcated flexible catheter in two different arteries. Other embodiments may use a single flexible catheter in a single artery.

In various embodiments, the device also may include an evacuation catheter, which is a second flexible catheter running parallel to the main catheter that may terminate near or just proximal to the vessel blocking mechanism. In various embodiments, the evacuation catheter may be coupled to the first flexible catheter. In various embodiments, the evacuation catheter may be used to flush debris from the area proximal to the vessel blocking mechanism, should any potential emboli, thrombi, or other debris accumulate during or after a TAVI or other procedure.

In various embodiments, the guidewire may be a TAD™ guidewire (Covidien, Mansfield Mass.) or another wire having a diameter of about 0.03-0.04 inches, such as about 0.035 inches. In some embodiments, the guidewire may have a tapered tip. Some embodiments of the embolism protection device may be used in conjunction with a concentric wire catheter guidewire system, such as disclosed in U.S. Pat. No. 7,402,141. For instance, a system of three or more concentric wires may be used to deploy the embolism protection device in a desired location. In various embodiments, a three-wire system may be used, with an inner wire that may serve as a guidewire, a second or middle wire that may be coupled to or carry one or more tools, such as surgical tools, that may be used during a procedure, and a third or outer wire that may serve to maintain the one or more surgical tools in a collapsed position until it has been positioned in a desired location.

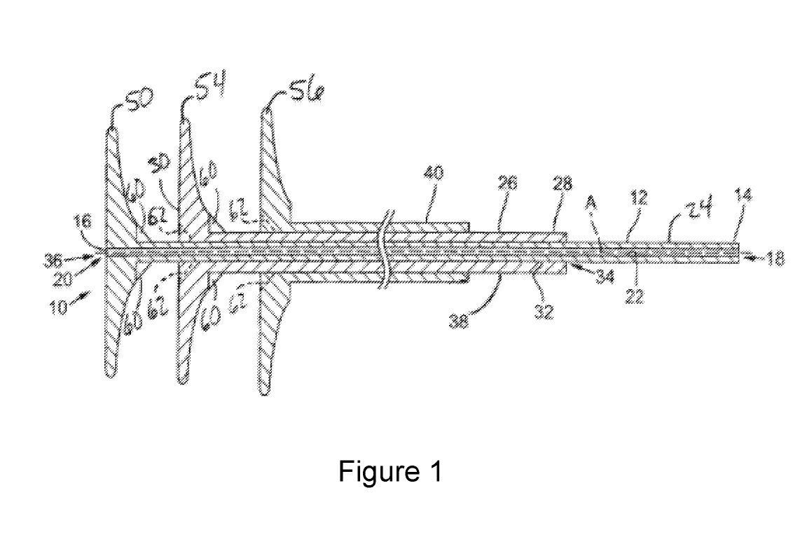

As shown in FIG. 1, an embodiment of the guidewire used to deploy the embolism protection device disclosed herein may include a multiple concentric wire system, indicated generally at 10. In various embodiments, system 10 may include an inner wire 12 having a distal end 14 and a proximal end 16. Inner wire 12 may have a length that may be selected for a particular type of procedure to be conducted in a human blood vessel, e.g., between about 150 cm and about 300 cm. Inner wire 12 may include an opening 18 adjacent distal end 14 and an opening 20 adjacent proximal end 16, and a central lumen 22 extending between the proximal and distal openings. In various embodiments, central lumen 22 may define an inner diameter for wire 12, and wire 12 also may have a generally cylindrical outer surface 24 defining an outer diameter. Typically, the outer diameter of inner wire 12 may be between about 0.004 and 0.018 inches, and may be any size therebetween, or larger or smaller as selected for the desired procedure and for compatibility with other wires, catheters, sheaths, and other equipment. For instance, the outer diameter of inner wire 12 may be 0.010, 0.014, or 0.018 inches in specific, non-limiting examples.

Optionally, inner wire 12 may be provided with a handle 50, which may be removable adjacent proximal end 16, so that it may be used by the physician in manipulating the wire about and along a central axis A of the wire. In some embodiments, wire 12 may be constructed without transitions between sections, if it includes any sections, of the wire. Inner wire 12 also may be used in crossing a bifurcation in the vessel, and thus may be provided with a rigidity selected to allow the bifurcation crossing. In some embodiments, rigidity may be controlled by the use of braiding or the selection of various materials. For example, nitinol may be flexible, but it may become stiffer as more stainless steel is added.

A second wire 26, which may be constructed to be deployed over inner wire 12, may include a distal end 28 and a proximal end 30 and a length preferably selected to be compatible with inner wire 12. In various embodiments, a central lumen 32 of wire 26 may extend between a distal opening 34 and a proximal opening 36. As described in greater detail below, second wire 26 may couple to the embolism protection device such that the device may be advanced along inner wire 12 and selectively deployed when in a desired position.

Central lumen 32 of second wire 26 may define an inner diameter for the wire, and second wire 26 may have a generally cylindrical outer surface 38 defining an outer diameter. In various embodiments, the outer diameter of wire 26 may be between about 0.008 and 0.035 inches, for instance about 0.025 inches or about 0.035 inches in specific, non-limiting examples, and may be any size therebetween, or larger or smaller as selected for the desired procedure and for compatibility with other wires, catheters, sheaths, and other equipment.

Optionally, wire 26 may be provided with a handle 54, which may be removable, adjacent proximal end 30 that the physician may use in manipulating the wire about and along a central axis A of the wire. In some embodiments, second wire 26 may have a rigidity selected to be greater than that of inner wire 12, thus providing the system with an overall variable rigidity which may depend on the extent to which inner wire 12 extends out of second wire 26.

System 10 may also include a third or outer wire 40 having proximal and distal ends with openings and a central lumen communicating therebetween, inner and outer diameters, and a generally cylindrical outer surface as for the other wires. In some embodiments, third wire 40 may be sized to fit over second wire 26, and optionally may include a handle 56 that may be removably coupled adjacent the proximal end for manipulation of the third wire about and along central axis A. In some embodiments, third wire 40 may have a rigidity selected to be greater than the rigidity of first wire 12 and/or greater than the rigidity of second wire 26, thus providing the system with an overall variable rigidity which depends on the extent to which inner wire 12 extends out of second wire 26, and the extent to which second wire 26 extends out of third wire 40.

Third wire 40 may have an outer diameter of between about 0.010-inches and about 0.064 inches, and may be any size therebetween, or larger or smaller as selected for the desired procedure and for compatibility with other wires, catheters, sheaths, and other equipment. For instance, in specific, non-limiting examples, third wire 40 may have an outer diameter of 0.035 or 0.064 inches. Typically, the length of third wire 40 may be less than the length of second wire 26, and the length of second wire 26 may be less than that of inner wire 12.

In one specific, non-limiting example of a suitable concentric wire system, first wire 12 may have an outer diameter of about 0.014 inches, second wire 26 may have an outer diameter of about 0.025 inches, and third wire 40 may have an outer diameter of about 0.035 inches. In various embodiments, such a concentric wire system may be compatible with a 4 French catheter system. In another specific, nonlimiting example, first wire 12 may have an outer diameter of about 0.018 inches, second wire 26 may have an outer diameter of about 0.035 inches, and third wire 40 may have an outer diameter of about 0.064 inches. In another specific, non-limiting example, first wire 12 may have an outer diameter of about 0.010 inches, second wire 26 may have an outer diameter of about 0.035 inches, and third wire 40 may have an outer diameter of about 0.064 inches. In various embodiments, these concentric wire systems may be compatible with a 5 or 6 French catheter system. Larger catheters also may be used, for example with an 8 French system.

In some embodiments, when the first, second, and third wires are coupled together, any of the handles of the first, second, and third wires, if present, may be used to manipulate all three wires, and also the wires may be manipulated relative to one another by simultaneous use of two or three of the handles. In some embodiments, a single handle may be used with system 10, and may be coupled to either first wire 12 or third wire 40.

In various embodiments, the length of first wire 12 may be between about 150 cm and about 300 cm, but may be other sizes as desired for particular procedures. Typically, the length of second wire 26 may be about 5 cm less than first wire 12, and the length of third wire 40 may be about 5 cm less than second wire 26.

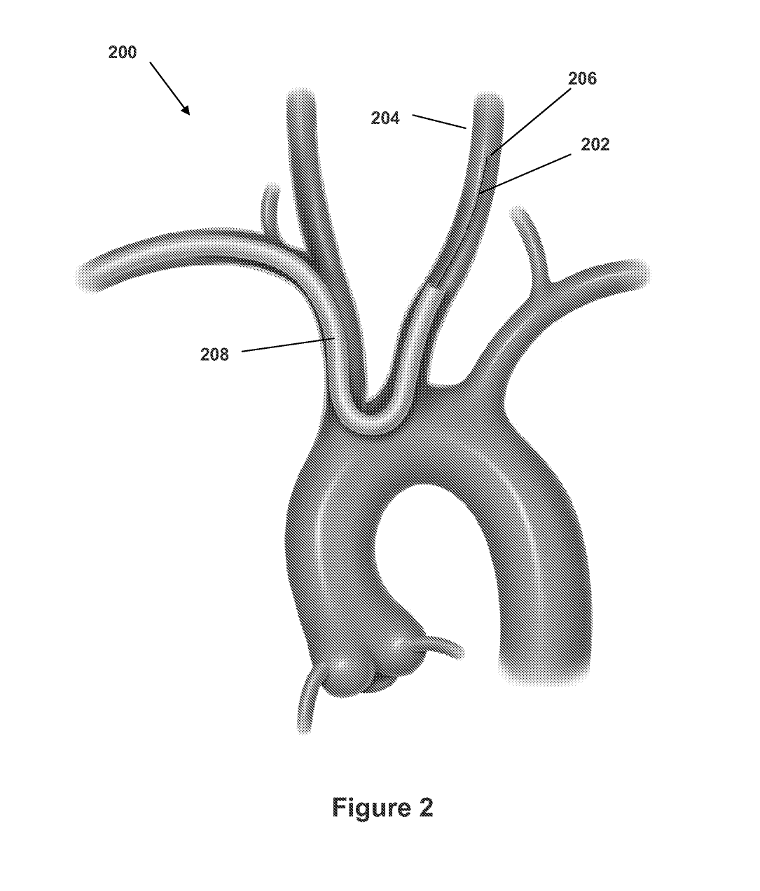

An embodiment of the embolism protection device is depicted in FIG. 2, in which an embolism protection system is indicated generally by reference number 200. System 200 may include a transporting mechanism, such as a guidewire 202, that is movable within a vessel 204, and that may correspond to first wire 12 in FIG. 1 in some examples. The vessel may be a vein, an artery, or may form part of the urinary, renal, or other fluid-transporting systems within a body. Specific, non-limiting examples of suitable arteries include the common or internal carotid artery, vertebral artery, innominate artery, and aorta. The embodiment shown in the figures relates specifically to a vein or artery having blood flowing therethrough.

In various embodiments, guidewire 202 may be inserted into the vasculature of a subject through any route known to those of skill in the art, such as by passing it through a femoral, brachial, or radial arterial route, and may be advanced to a desired location, such as an innominate or carotid artery. In some embodiments, guidewire 202 may have a tapered distal end 206, and may be sized and configured to permit a sheath 208 to be threaded thereupon and advanced to a desired location. In specific, non-limiting embodiments, sheath 208 may be a 6 French, 8 French, or 10 French sheath.

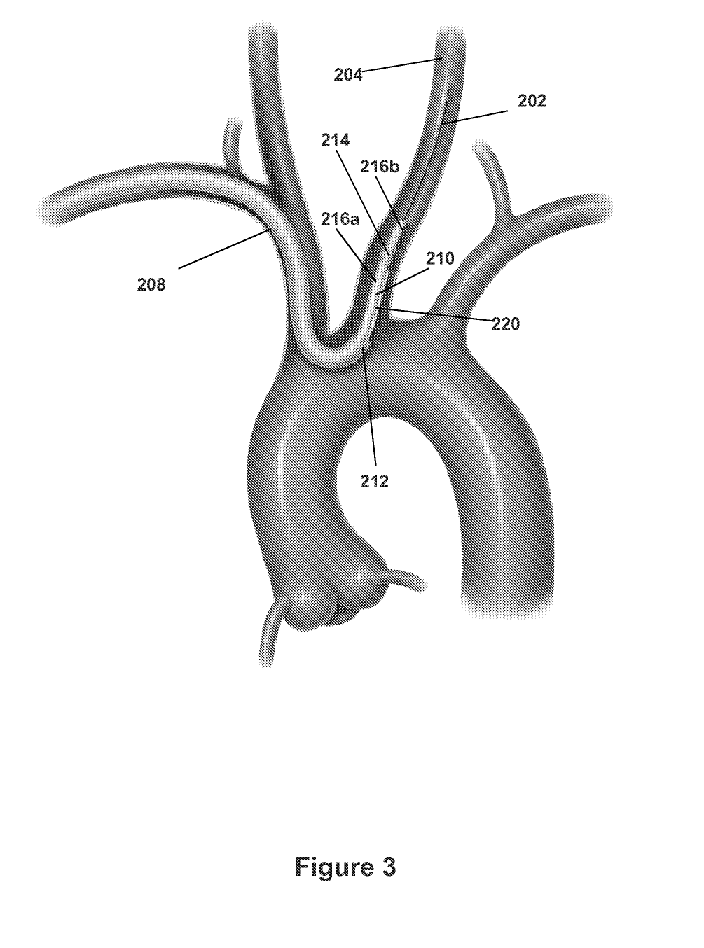

Turning now to FIG. 3, once sheath 208 has been advanced along guidewire 202, a flexible catheter 210 may be advanced over guidewire 202 and through sheath 208, and may be passed through the aperture at the distal end 212 of sheath 208. In various embodiments, flexible catheter 210 may include an expandable membrane 214 (e.g., a balloon) coupled to its exterior surface. In various embodiments, expandable membrane 214 may be configured such that it may be inflated by a user in order to block blood flow through vessel 204. In various embodiments, expandable membrane 214 also may be coupled to a locking mechanism (not shown) that may maintain expandable membrane 214 in an inflated state during an interventional procedure, such as a TAVI.

In various embodiments, the size of flexible catheter 210 may range from about 4 French to about 8 French, depending on the size and age of the patient, the artery selected for treatment, and route of entry into the vasculature (e.g., a radial approach will require a smaller diameter catheter than a femoral approach). In various embodiments, flexible catheter may include a straight or curved portion at the distal end, so as to facilitate navigation of the device to a desired position in the vasculature. For example, in specific, non-limiting examples, flexible catheter 210 may have a diameter and curve similar to that of any of various commercially available guiding catheters, such as the Torcon NB™ Advantage catheter (5 French) or the Shuttle Select Slip-Cath™ (6 French), both from Cook Incorporated (Bloomington, Ind.); The Vistabritetip™ (7 French) from Cordis (Tipperary, Ireland); the Launcher™ LCB (7 French) or 3DRC (8 French) from Medtronic (Minneapolis, Minn.); or the Glidecath™ straight (4 French), Heartrail III™ Ikari Right (6 French), the Radiofocus Optitorque™ Jacky Radial (5 French), or the Radiofocus Optitorque™ Radial TIG 4.0 (5 French) from Terumo Corporation (Somerset, N.J.).

In various embodiments, flexible catheter 210 also may include a plurality of perforations 216 both proximal 216a and distal 216b to expandable membrane 214. In various embodiments, these perforations may provide a bypass conduit past expandable membrane 214, so that some blood may still flow past the vessel blocking mechanism when expandable membrane 214 is expanded and blocking vessel 204. In some embodiments, these perforations may have an average diameter of about 20-350 microns, such as 40-325 microns, or about 50-300 microns.

Although expandable membrane 214 may assume a variety of shapes in order to meet the needs of the specific application, in various embodiments it may assume a generally cylindrical shape about the circumference of flexible catheter 210, and it may be made from a very compliant material, such that when expanded, expandable membrane 214 may conform to the contours of vessel 204, and it may form an atraumatic occlusion in vessel 204. In various embodiments, when expanded, expandable membrane 214 may assume a generally elliptical or circular shape against the wall of vessel 204. In various embodiments, expandable membrane 214 may have an inflated diameter of from about 10 mm to about 20 mm. In some embodiments, this diameter may be chosen to suit a particular use. For example, when system 200 is to be used in the innominate artery, expandable membrane 214 may have an expanded diameter of from about 15 mm to about 20 mm. When system 200 is to be used in the carotid artery, it may have a diameter of from about 10 mm to about 15 mm, for example about 12 mm to about 14 mm.

Thus, in various embodiments, when system 200 is thus positioned and expandable membrane 214 is thus expanded, system 200 may allow some blood to bypass the occlusion created by expandable membrane 214 via perforations 216 in flexible catheter 210, thereby preventing ischemia, while also trapping and containing potential emboli and thrombi that are larger than perforations 216, thus preventing downstream tissue damage. Once the TAVI (or other) procedure has been completed, evacuation catheter 220 may be used to flush out any debris or potential emboli or thrombi that may have settled proximal to expandable membrane 214 as a result of the TAVI (or other) procedure. In specific, nonlimiting examples, evacuation catheter 220 may be a Pronto™ catheter (Vascular Solutions, Minneapolis, Minn.) or an Export™ Catheter (Medtronic, Minneapolis, Minn.), or another commercially available catheter of suitable dimensions. In some embodiments, evacuation catheter 220 may be coupled to flexible catheter 210, whereas in other embodiments, the two catheters may be separate. In particular examples, evacuation capabilities may be provided not as a separate catheter, but as a separate lumen in flexible catheter 210.

In various embodiments, once the area proximal to expandable membrane 214 is free of debris and possible emboli and thrombi, expandable membrane 214 may be deflated, and flexible catheter 210 and guidewire 202 may be removed.

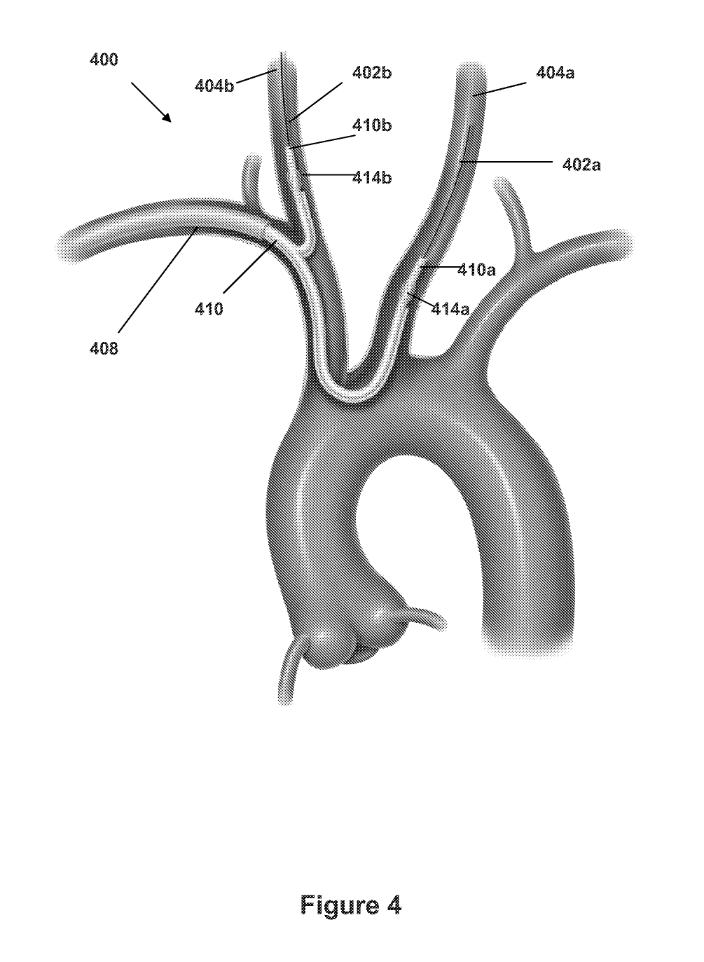

Another embodiment is illustrated in FIG. 4, which depicts a bifurcated embolism protection device that may be used to protect the vasculature on both sides of the body, such as in both carotid arteries, both innominate arteries, or an innominate artery and a carotid artery. In the illustrated embodiment, after placing a first guidewire 402a and a first arm 410a of bifurcated flexible catheter 410) in a desired location within a first vessel 404a essentially as described above with regard to FIGS. 2 and 3, sheath 408 is withdrawn, revealing a second arm 410b of bifurcated flexible catheter 410 through which a second guidewire 402b is passed into a desired location in a second vessel 404b Like first guidewire 402a, second guidewire 402b may be a TAD™ guidewire (Covidien, Mansfield Mass.) or another wire having a diameter of about 0.03-0.04 inches, such as about 0.035 inches. In some embodiments, guidewire 402b may have a tapered tip, and/or may be a concentric wire such as is illustrated in FIG. 1.

Second arm 410b of bifurcated flexible catheter 410 is then positioned over second guidewire 402b in a desired location in a second vessel 404b. Like first farm 410a, in various embodiments, second arm 410b of bifurcated flexible catheter 410 may include a second expandable membrane 414b (e.g., a balloon) coupled to its exterior surface. In various embodiments, second expandable membrane 414b may be configured such that it may be inflated by a user in order to block blood flow through second artery 404b. In various embodiments, second expandable membrane 414b also may be coupled to a locking mechanism (not shown) that may maintain second expandable membrane 414b (or both first and second expandable membranes 414a, 414b) in an inflated state during a coronary procedure, such as a TAVI.

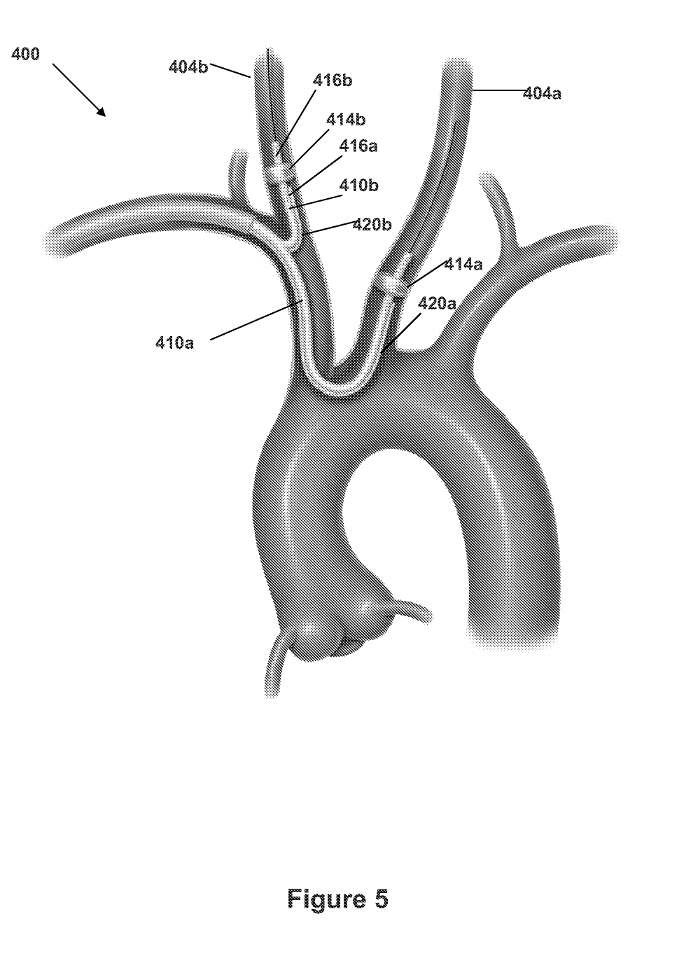

FIG. 5 illustrates system 400 of FIG. 4 with expandable membranes 414a, 414b in an inflated state and obstructing first and second vessels. Like first arm 410a, in various embodiments, second arm 410b also may include a plurality of perforations 416 both proximal 416a and distal 416b to second expandable membrane 414b. In various embodiments, these perforations may provide a bypass conduit past second expandable membrane 414b, so that some blood may still flow when second expandable membrane 414b is expanded and blocking second vessel 404b.

Although second expandable membrane 414b may assume any of a variety of shapes in order to meet the needs of the specific application, in various embodiments it may assume a generally cylindrical shape. In various embodiments, expandable membrane 414b may have an inflated diameter that is the same as or different from that of first expandable membrane 414a. In various embodiments, second expandable membrane 414b may have an expanded diameter of from about 10 mm to about 20 mm, and this diameter may vary independently of the expanded diameter of first expandable membrane 414a in various embodiments. In some embodiments, first and second expandable membranes 414a, 414b may be expanded simultaneously in a single inflation step.

Thus, in various embodiments, when system 400 is thus positioned, and expandable membranes 414a, 414b are thus expanded, system 400 may allow some blood to bypass the occlusions created by expandable membranes 414a, 414b via perforations 416 in the first and second arms 410a, 410b of flexible catheter 410, thereby preventing ischemia, while also trapping and containing potential emboli and thrombi, thus preventing downstream tissue damage. Once the TAVI (or other) procedure has been completed, evacuation catheters 420a, 420b may be used to flush out any debris or potential emboli that may have settled proximal to expandable membranes 414a, 414b as a result of the TAVI (or other) procedure. In various embodiments, once the area proximal to each expandable membrane 414a, 414b is free of debris and possible emboli and thrombi, expandable membranes 414a, 414b may be deflated, and bifurcated flexible catheter 410 and guidewires 402a, 402b may be removed.

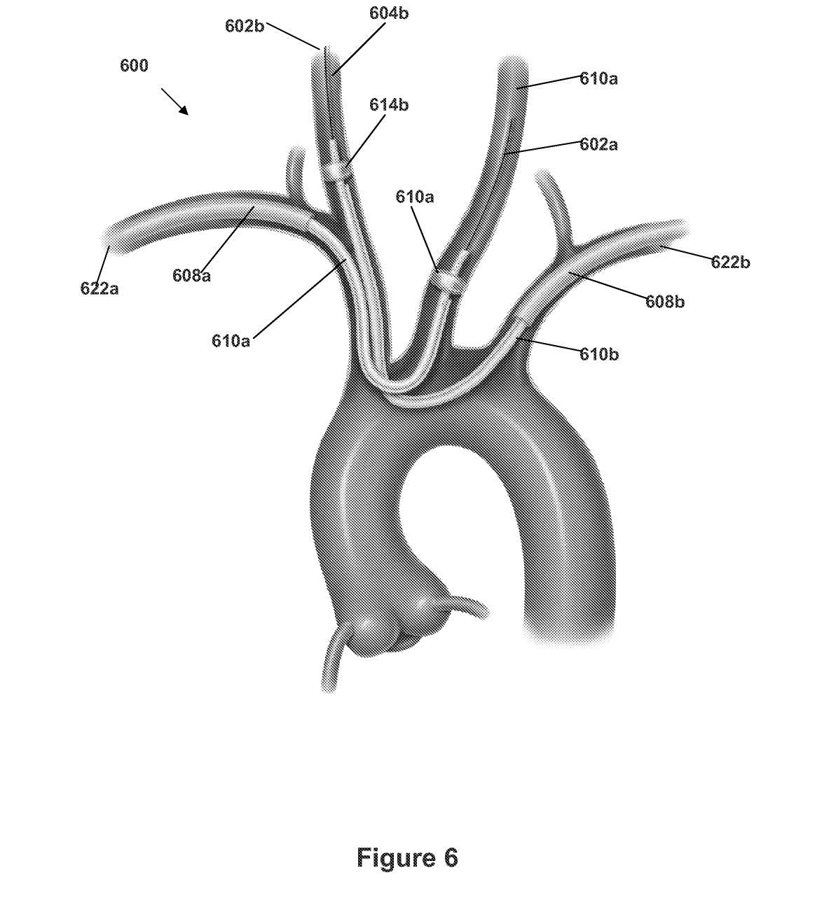

FIG. 6 illustrates an alternate embodiment of the system 600 wherein two flexible catheters 610a, 610b, each with an expandable membrane 614a, 614b coupled thereto, may be inserted sequentially into first and second vessels 604a, 604b over first and second guidewires 602a, 602b and through first and second sheaths 608a, 608b via two different access routes 622a, 622b. In one specific, non-limiting example, one flexible catheter may be inserted via the right radial or brachial artery in order to access the left carotid artery, and the other flexible catheter may be inserted via the left radial or brachial artery in order to access the innominate or right common carotid artery.

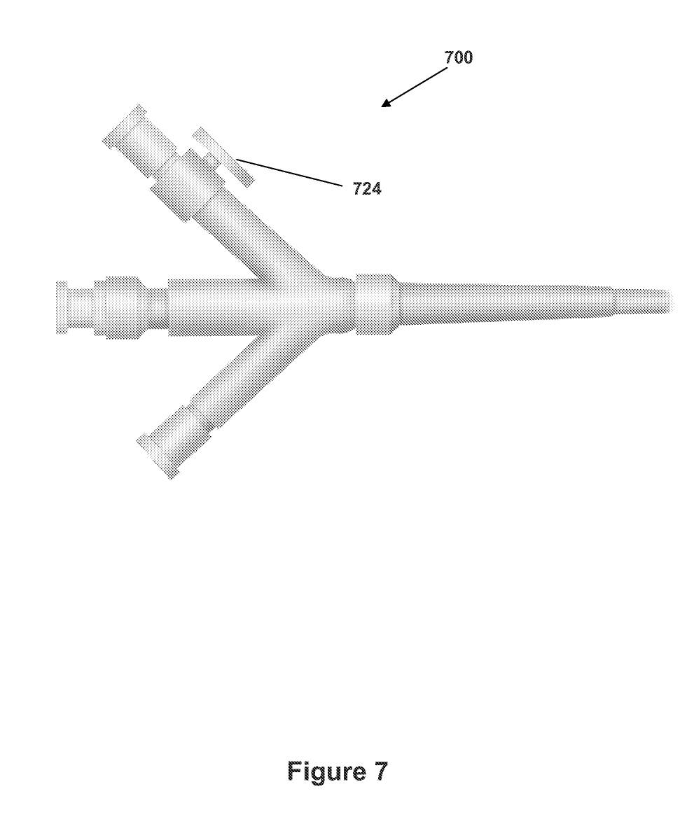

FIG. 7 illustrates a port 700 through which one or more guidewires (not shown) may be inserted and controlled by a user. In various embodiments, one arm of port 700 may be used for passing the one or more guidewires, one may be used for inflating the expandable membrane, and one may be used for applying suction through the evacuation catheter. In various embodiments, port 700 may include a locking mechanism 724 that may be actuated in order to maintain one or more expandable membranes (not shown) in an inflated state during a TAVI or other procedure.

In one specific, non-limiting example, the embolism protection device may be used to protect the brain of a subject undergoing a TAVI procedure or other percutaneous valve procedure. In this example, the flexible catheter may be up to 6 French in size, and two embolism protection devices are placed in different arteries. First two guidewires each having a length of about 150 cm are placed radially or brachially. In one specific, non-limiting example, the first guidewire is passed either into the right innominate artery or into the left carotid artery via the right radial artery. The second guidewire is placed via the left radial artery and passed into the innominate artery or the right common carotid artery. In this specific, non-limiting example, a flexible catheter having an expandable membrane as described above is advanced over each guidewire into a desired position.

In particular examples, a device placed in the innominate artery may have an expandable membrane with an expanded diameter of between about 15 mm and about 20 mm. In particular examples, a device placed in the left carotid artery may have an expandable membrane having an expanded diameter of about 10 mm to about 14 mm. After each device is placed, both are simultaneously inflated and locked with a locking mechanism. As described above, each flexible catheter includes a plurality of small holes just proximal and distal of the expandable membrane (e.g., the vessel blocking mechanism). These holes may allow sufficient blood to bypass the vessel blocking mechanism to prevent ischemia during the procedure. After the procedure is completed, the user may apply suction to the area proximal to each expandable membrane, for instance with a Pronto™ or Export™ catheter, to remove any potential emboli or thrombi that were dislodged during the TAVI procedure. Once the area proximal to the vessel blocking mechanism is clear of debris, the expandable membrane may be deflated and the device removed from the vasculature.

In another specific, non-limiting example, a bifurcated flexible catheter may be used to protect two different arteries with a single device. In various embodiments, each arm of the bifurcated flexible catheter may have a diameter of from about 5 French to about 8 French, and it may be placed via the right brachial artery or right radial artery. In various embodiments, the bifurcated flexible catheter may be advanced over a guidewire essentially as described above within a larger sheath that is sized to accommodate both arms of the bifurcated flexible catheter. From the right brachial artery or right radial artery, the bifurcated flexible catheter may be passed up into the left common carotid artery. In various embodiments, as the bifurcated flexible catheter is placed into the left common carotid artery, the sheath may be retracted to express a fairly rigid right common carotid limb (e.g., a second arm of the birfurcated flexible catheter) is expressed. In various embodiments, with the limb expressed, a second guidewire may be passed inside that limb (if necessary) in order to access the right common carotid artery. In various embodiments, a single inflation may be performed that may dilate and inflate both the first and second expandable membranes (e.g., vessel blocking mechanisms) on the first and second arms of the bifurcated flexible catheter. In various embodiments, some blood may bypass the vessel blocking mechanisms via a plurality of perforations in the flexible catheter as described above. The TAVI (or other) procedure may then be performed. In various embodiments, following this procedure, suction may be applied to the area just proximal to each expandable membrane via the first and second evacuation catheters. In various embodiments, any material suctioned out may then be placed through a fine filter. In various embodiments, if filtration reveals no clots or plaque materials, the expandable membranes may be deflated and the device may be removed.

In another specific, non-limiting example, the patient may be prepared for a TAVI procedure with the groin prepped. In various embodiments, both the right and left radial and/or brachial arteries also may be prepped. In various embodiments, a Vitek™ or right coronary catheter may be passed over a guidewire via the right brachial artery or radial artery. In various embodiments, once the Vitek™ catheter has gained access to the left common carotid artery, a first flexible catheter (with an expandable membrane coupled thereto as described above) may be passed into the left common carotid artery.

Next, in various embodiments, the left radial or brachial artery may be accessed with a similar approach using a guidewire and a Vitek™ catheter, both of which may be passed into the innominate artery and then into the right common carotid artery. In various embodiments, a second flexible catheter (with and expandable membrane coupled thereto) may be passed into the right common carotid artery. In various embodiments, once both devices have been placed, both the first and second expandable membranes may be inflated and locked in the inflated position. In some embodiments, an angiogram may be performed in order to verify adequate perfusion through the innominate or carotid artery, for example using a Touchyboarst injector to inject dye through or around the wire distal to the lumen. In various embodiments, this dye may be visualized flowing into both carotid arteries through the perforations proximal and distal to the vessel blocking mechanism, confirming maintenance of some blood flow past each occlusion. In various embodiments, the TAVI procedure may then be performed via either a femoral or apical approach. In various embodiments, suction may be applied to the evacuation catheters, for example via a standard locking syringe. In various embodiments, the material suctioned through the evacuation catheters may then be flushed through a filter, and once the filter shows complete clearing of all debris in the suctioned material, both expandable membranes may be deflated. In some embodiments, an angiogram may be performed to verify that blood flow is adequate following the procedure.

Although certain embodiments have been illustrated and described herein, it will be appreciated by those of ordinary skill in the art that a wide variety of alternate and/or equivalent embodiments or implementations calculated to achieve the same purposes may be substituted for the embodiments shown and described without departing from the scope. Those with skill in the art will readily appreciate that embodiments may be implemented in a very wide variety of ways. This application is intended to cover any adaptations or variations of the embodiments discussed herein. Therefore, it is manifestly intended that embodiments be limited only by the claims and the equivalents thereof.

What is claimed is:

1. An intravascular embolism protection device comprising:
a first wire having a proximal end, a distal end, and an outer diameter;
a second wire having a proximal end, a distal end, and an outer diameter;
a bifurcated flexible catheter having a proximal end, a first arm having a first outer surface and a first distal end, a second arm having a second outer surface and a second distal end, and an inner lumen sized at the proximal end to accommodate the outer diameter of both the first and second wires;
wherein the first arm comprises:
a first vessel blocking mechanism mounted on the first outside surface, the first vessel blocking mechanism configured to block a first vessel;
a first fluid bypass channel configured to permit fluid to bypass a designated region of the first vessel when the first blocking mechanism blocks the first vessel; and
a first evacuation catheter configured to remove debris from the first vessel while the first vessel blocking mechanism blocks the first vessel; and
wherein the second arm comprises:
a second vessel blocking mechanism mounted on the second outside surface, the second vessel blocking mechanism configured to block a second vessel;
a second fluid bypass channel configured to permit fluid to bypass a designated region of the second vessel when the second blocking mechanism blocks the second vessel; and
a second evacuation catheter configured to remove debris from the second vessel while the second vessel blocking mechanism blocks the second vessel.

2. The intravascular embolism protection device of claim 1, wherein the first and second vessel blocking mechanisms are configured to have a collapsed state and an expanded state.

3. The intravascular embolism protection device of claim 1, wherein the first vessel blocking mechanism couples to the first arm near the first distal end, and wherein the second vessel blocking mechanism couples to the second arm near the second distal end.

4. The intravascular embolism protection device of claim 3, wherein the first and second vessel blocking mechanisms each comprise a compliant expandable membrane.

5. The intravascular embolism protection device of claim 4, wherein the compliant expandable membrane has an expanded diameter of from about 10 mm to about 20 mm.

6. The intravascular embolism protection device of claim 5, wherein the compliant expandable membrane has an expanded diameter of from about 15 mm to about 20 mm.

7. The intravascular embolism protection device of claim 5, wherein the compliant expandable membrane has an expanded diameter of from about 12 mm to about 14 mm.

8. The intravascular embolism protection device of claim 1, wherein the first and second fluid bypass channels comprise perforations in the first and second arms that are sized to permit blood to pass through without allowing emboli to pass through.

9. The intravascular embolism protection device of claim 8, wherein the perforations have an average diameter of from about 50 microns to about 300 microns.

10. The intravascular embolism protection device of claim 8, wherein the perforations are positioned on the first and second arms both proximal and distal to the first and second vessel blocking mechanism.

11. The intravascular embolism protection device of claim 1, wherein the device further comprises:
a second wire having a proximal end, a distal end, an inner lumen, and an outer diameter, the inner lumen sized to accommodate the outer diameter of the first wire; and
a third wire having a proximal end, a distal end, and an inner lumen, the inner lumen sized to accommodate the outer diameter of the second wire.

12. A method of protecting a subject from embolism during a percutaneous valve procedure, comprising:
advancing a first wire through the vasculature of the subject to a first artery;
advancing a second wire having a proximal end, a distal end, and an outer diameter through the vasculature of the subject to a second artery;
advancing a first flexible catheter over the first wire to the first artery;
advancing a second flexible catheter over the second wire to the second artery;
wherein the first and second flexible catheters each comprise:
a vessel blocking mechanism mounted on an outside surface of the flexible catheter, the vessel blocking mechanism configured to block a vessel;
a fluid bypass channel configured to permit fluid to bypass a designated region of the vessel when the blocking mechanism blocks the vessel; and
an evacuation catheter configured to remove debris from the vessel while the vessel blocking mechanism blocks the vessel;
activating the vessel blocking mechanisms to block the first and second arteries;
performing the percutaneous valve procedure;
applying suction to the evacuation catheters to remove potential emboli and/or thrombi from the first and second arteries;
de-activating the vessel blocking mechanisms; and
removing the first and second flexible catheters and first and second wires from the vasculature of the subject.

13. The method of claim 12, wherein the vessel blocking mechanisms comprise compliant expandable membranes and wherein activating the vessel blocking mechanisms comprises inflating the expandable membranes to occlude the first and second arteries.

14. The method of claim 12, wherein the first and second arteries are innominate or carotid arteries.

15. The method of claim 12, wherein the fluid bypass channels comprise perforations in the first and second flexible catheters that are sized to permit blood to pass through without allowing emboli to pass through.

16. The method of claim 15, wherein the perforations have an average diameter of from about 50 microns to about 300 microns and are positioned on the first and second flexible catheters both proximal and distal to the vessel blocking mechanisms.

17. A method of protecting a subject from embolism during a percutaneous valve procedure, comprising:
advancing a first wire through the vasculature of the subject to a first artery;
advancing a sheath over the first wire through the vasculature of the subject to the first artery;
advancing a first arm of a bifurcated flexible catheter over the first wire to the first artery, wherein the bifurcated flexible catheter comprises a proximal end, the first arm having a first distal end, and a second arm having a second distal end, and wherein the first and second arms each comprise:
a vessel blocking mechanism mounted on an outside surface of the arm, the vessel blocking mechanism configured to block a vessel;
a fluid bypass channel configured to permit fluid to bypass a designated region of the vessel when the blocking mechanism blocks the vessel; and
an evacuation catheter configured to remove debris from the vessel while the vessel blocking mechanism blocks the vessel;
retracting the sheath to cause the second arm of the bifurcated flexible catheter to deploy;
optionally advancing a second wire through the second distal end of the second arm of the bifurcated flexible catheter to help direct the second arm to a second artery;
directing the second arm to the second artery;
activating the vessel blocking mechanisms to block the first and second arteries;
performing the percutaneous valve procedure;
applying suction to the evacuation catheters to remove potential emboli and/or thrombi from the first and second arteries;
de-activating the vessel blocking mechanisms; and
removing the bifurcated flexible catheter and first and second wires from the vasculature of the subject.

18. The method of claim 17, wherein the vessel blocking mechanisms comprise compliant expandable membranes, and wherein activating the vessel blocking mechanisms comprises inflating the compliant expandable membranes to occlude the first and second arteries.

19. The method of claim 17, wherein the first and second arteries are innominate or carotid arteries.

20. The method of claim 17, wherein the fluid bypass channels comprise perforations in the first and second arms that are sized to permit blood to pass through without allowing emboli to pass through.

21. The method of claim 20, wherein the perforations have an average diameter of from about 50 microns to about 300 microns and are positioned on the first and second flexible catheters both proximal and distal to the vessel blocking mechanisms.
